(19) United States
(12) Patent Application Publication
(10) Pub. No.: US 2025/0263499 A1
(43) Pub. Date: Aug. 21, 2025

(54) ANTI-CD24 ANTIBODY AND USE THEREOF

(71) Applicant: SHENGHE (CHINA) BIOPHARMACEUTICAL CO., LTD., Nanjing, Jiangsu (CN)

(72) Inventors: Xiaoling JIANG, Nanjing (CN); Chongbing WU, Nanjing (CN); Liusong YIN, Nanjing (CN)

(73) Assignee: SHENGHE (CHINA) BIOPHARMACEUTICAL CO., LTD., Nanjing, Jiangsu (CN)

(21) Appl. No.: 18/576,215

(22) PCT Filed: Jun. 30, 2022
(86) PCT No.: PCT/CN2022/102729

§ 371 Date: Jan. 3, 2024

(30) Foreign Application Priority Data
Jul. 6, 2021 (WO) PCT/CN2021/104623
Sep. 24, 2021 (WO) PCT/CN2021/120110

(51) Int. Cl. C07K 16/28; A61P 35/00

(52) U.S. Cl. CPC C07K 16/2896; A61P 35/00; C07K 2317/24; C07K 2317/92

(57) ABSTRACT

An anti-CD24 antibody or antigen-binding fragment thereof capable of binding to CD24, and/or capable of exerting an anti-tumor effect through an antibody Fc fragment, and use thereof.

### TECHNICAL FIELD

The present invention belongs to the field of tumor immunotherapy and molecular immunology and specifically relates to an anti-CD24 antibody or antigen-binding fragment thereof.

### BACKGROUND

With the development of a deeper understanding of the immune system and the mechanisms of tumorigenesis, tumor immunotherapy has increasingly become a powerful weapon against tumors.

Tumor-targeted monoclonal antibodies are one of the most important tools in the field of tumor immunotherapy. Macrophage phagocytosis requires the simultaneous action of two signals: activation of the “eat me” signal on the surface of the target cell, and inactivation of the “don't eat me” signal on the surface of the same cell. The absence of either signal is not sufficient to trigger phagocytosis. There is increasing evidence that CD47 is a class of “don't eat me” signals, and that tumor cells that highly express CD47 release “don't eat me” signals by binding to signal-regulated protein α (SIRPα) on the surface of macrophages, thus preventing tumor cells from being phagocytosed by macrophages.

CD24, also known as heat-stable antigen, is a highly glycosylated glycosylphosphatidylinositol-anchored surface protein. It has been found that CD24 signaling is often used as a complementary pathway to CD47 signaling in cancers such as blood cancers, which are very susceptible to CD47 signaling blockade and insensitive to CD24 signaling blockade, whereas in other cancers such as ovarian cancers, the opposite is true, which allows the majority of cancers to be attacked by blocking one of these signals. In addition, cancers may be more susceptible to attack if multiple “don't eat me” signals are blocked on the tumor, and of the many primary and secondary “don't eat me” signals, CD24 is the most prominent one. It has been shown that CD24 is used by cancer cells to protect themselves by binding to Siglec-10 on the surface of macrophages and activating the SHP-1/SHP-2-mediated inhibitory signaling pathway, which is a very promising target for the development of cancer immunotherapy. High CD24 expression has been clinically shown to be negatively correlated with the prognosis of breast cancer. The development of anti-CD24 antibodies could target refractory ovarian cancer and triple-negative breast cancer.

In conclusion, CD24 is an anti-phagocytic signal that is highly expressed in a variety of cancers, and anti-CD24 antibodies have a solid theoretical basis in efficacy and can increase the chemosensitivity of cancer cells, blocking CD24 and macrophage surface Siglec-10 binding, disarming the inhibitory signaling to tumor cells is expected to be a novel immunotherapy.

### SUMMARY

The present invention provides an anti-CD24 antibody or antigen-binding fragment thereof, wherein the antibody or antigen-binding fragment thereof has at least one of the following properties:
(1) capable of binding to CD24; and
(2) capable of exerting an anti-tumor effect through the antibody Fc fragment.

In some embodiments, the anti-CD24 antibody or antigen-binding fragment comprises a heavy chain variable region and a light chain variable region, wherein the heavy chain variable region comprises the heavy chain complementary determining regions HCDR1, HCDR2, and HCDR3, and the light chain variable region comprises the light chain complementary determining regions LCDR1, LCDR2, and LCDR3, wherein
(a) HCDR1 of the heavy chain variable region is selected from any amino acid sequence of SEQ ID NOs: 2, 17, or a sequence having at least 80%, 85%, 90%, 91%, 92%, 93%, 94%, 95%, 96%, 97%, 98%, 99% or more identity to any amino acid sequence of SEQ ID NOs: 2, 17, or an amino acid sequence having one or more (preferably 2 or 3) conserved amino acid mutations (preferably substitutions, insertions, or deletions) in comparison to any amino acid sequence of SEQ ID NOs: 2, 17;
(b) HCDR2 of the heavy chain variable region is selected from any amino acid sequence of SEQ ID NOs: 3, 18, or a sequence having at least 80%, 85%, 90%, 91%, 92%, 93%, 94%, 95%, 96%, 97%, 98%, 99% or more identity to any amino acid sequence of SEQ ID NOs: 3, 18, or an amino acid sequence having one or more (preferably 2 or 3) conserved amino acid mutations (preferably substitutions, insertions, or deletions) in comparison to any amino acid sequence of SEQ ID NOs: 3, 18;
(c) HCDR3 of the heavy chain variable region is selected from any amino acid sequence of SEQ ID NOs: 4, 19, or a sequence having at least 80%, 85%, 90%, 91%, 92%, 93%, 94%, 95%, 96%, 97%, 98%, 99% or more identity to any amino acid sequence of SEQ ID NOs: 4, 19, or an amino acid sequence having one or more (preferably 2 or 3) conserved amino acid mutations (preferably substitutions, insertions, or deletions) in comparison to any amino acid sequence of SEQ ID NOs: 4, 19;
(d) LCDR1 of the light chain variable region is selected from any amino acid sequence of SEQ ID NOs: 6, 21, or a sequence having at least 80%, 85%, 90%, 91%, 92%, 93%, 94%, 95%, 96%, 97%, 98%, 99% or more identity to any amino acid sequence of SEQ ID NOs: 6, 21, or an amino acid sequence having one or more (preferably 2 or 3) conserved amino acid mutations (preferably substitutions, insertions, or deletions) in comparison to any amino acid sequence of SEQ ID NOs: 6, 21;
(e) LCDR2 of the light chain variable region is selected from any amino acid sequence of SEQ ID NOs: 7, 22, or a sequence having at least 80%, 85%, 90%, 91%, 92%, 93%, 94%, 95%, 96%, 97%, 98%, 99% or more identity to any amino acid sequence of SEQ ID NOs: 7, 22, or an amino acid sequence having one or more (preferably 2 or 3) conserved amino acid mutations (preferably substitutions, insertions, or deletions) in comparison to any amino acid sequence of SEQ ID NOs: 7, 22; and/or
(f) LCDR3 of the light chain variable region is selected from any amino acid sequence of SEQ ID NOs: 8, 23, or a sequence having at least 80%, 85%, 90%, 91%, 92%, 93%, 94%, 95%, 96%, 97%, 98%, 99% or more identity to any amino acid sequence of SEQ ID NOs: 8, 23, or an amino acid sequence having one or more (preferably 2 or 3) conserved amino acid mutations (preferably substitutions, insertions, or deletions) in comparison to any amino acid sequence of SEQ ID NOs: 8, 23;

In some embodiments, the anti-CD24 antibody or antigen-binding fragment, HCDR1, HCDR2, HCDR3 of the heavy chain variable region consist of the amino acid sequences SEQ ID NO: 2, SEQ ID NO: 3 and SEQ ID NO: 4, respectively, and LCDR1, LCDR2, LCDR3 of the light chain variable region consist of SEQ ID NO: 6, SEQ ID NO: 7 and SEQ ID NO: 8, respectively.

In some embodiments, the heavy chain variable region has the amino acid sequence given in SEQ ID NO: 1 and the light chain variable region that has the amino acid sequence given in SEQ ID NO: 5.

In some embodiments, the heavy chain variable region and light chain variable region are selected from a group of the following (1)-(12) amino acid sequence combinations:

| NO. | Heavy chain variable region | Light chain variable region |
|---|---|---|
| (1) | SEQ ID NO: 9 | SEQ ID NO: 13 |
| (2) | SEQ ID NO: 9 | SEQ ID NO: 14 |
| (3) | SEQ ID NO: 9 | SEQ ID NO: 15 |
| (4) | SEQ ID NO: 10 | SEQ ID NO: 13 |
| (5) | SEQ ID NO: 10 | SEQ ID NO: 14 |
| (6) | SEQ ID NO: 10 | SEQ ID NO: 15 |
| (7) | SEQ ID NO: 11 | SEQ ID NO: 13 |
| (8) | SEQ ID NO: 11 | SEQ ID NO: 14 |
| (9) | SEQ ID NO: 11 | SEQ ID NO: 15 |
| (10) | SEQ ID NO: 12 | SEQ ID NO: 13 |
| (11) | SEQ ID NO: 12 | SEQ ID NO: 14 |
| (12) | SEQ ID NO: 12 | SEQ ID NO: 15 |

In some embodiments, HCDR1, HCDR2, HCDR3 of the heavy chain variable region consist of the amino acid sequences SEQ ID NO: 17, SEQ ID NO: 18, and SEQ ID NO: 19, respectively, and LCDR1, LCDR2, LCDR3 of the light chain variable region consist of SEQ ID NO: 21, SEQ ID NO: 22, and SEQ ID NO: 23, respectively.

In some embodiments, the heavy chain variable region has the amino acid sequence given in SEQ ID NO: 16 and the light chain variable region has the amino acid sequence given in SEQ ID NO: 20.

In some embodiments, the heavy chain variable region and light chain variable region are selected from a group of the following (1)-(24) amino acid sequence combinations:

| NO. | Heavy chain variable region | Light chain variable region |
|---|---|---|
| (1) | SEQ ID NO: 24 | SEQ ID NO: 32 |
| (2) | SEQ ID NO: 24 | SEQ ID NO: 33 |
| (3) | SEQ ID NO: 24 | SEQ ID NO: 34 |
| (4) | SEQ ID NO: 25 | SEQ ID NO: 32 |
| (5) | SEQ ID NO: 25 | SEQ ID NO: 33 |
| (6) | SEQ ID NO: 25 | SEQ ID NO: 34 |
| (7) | SEQ ID NO: 26 | SEQ ID NO: 32 |
| (8) | SEQ ID NO: 26 | SEQ ID NO: 33 |
| (9) | SEQ ID NO: 26 | SEQ ID NO: 34 |
| (10) | SEQ ID NO: 27 | SEQ ID NO: 32 |
| (11) | SEQ ID NO: 27 | SEQ ID NO: 33 |
| (12) | SEQ ID NO: 27 | SEQ ID NO: 34 |
| (13) | SEQ ID NO: 28 | SEQ ID NO: 32 |
| (14) | SEQ ID NO: 28 | SEQ ID NO: 33 |
| (15) | SEQ ID NO: 28 | SEQ ID NO: 34 |
| (16) | SEQ ID NO: 29 | SEQ ID NO: 32 |
| (17) | SEQ ID NO: 29 | SEQ ID NO: 33 |
| (18) | SEQ ID NO: 29 | SEQ ID NO: 34 |
| (19) | SEQ ID NO: 30 | SEQ ID NO: 32 |
| (20) | SEQ ID NO: 30 | SEQ ID NO: 33 |
| (21) | SEQ ID NO: 30 | SEQ ID NO: 34 |
| (22) | SEQ ID NO: 31 | SEQ ID NO: 32 |
| (23) | SEQ ID NO: 31 | SEQ ID NO: 33 |
| (24) | SEQ ID NO: 31 | SEQ ID NO: 34 |

In some embodiments, the anti-CD24 antibody or antigen-binding fragment comprises a mouse-derived antibody or antigen-binding fragment thereof, a chimeric antibody or antigen-binding fragment thereof, and/or a humanized antibody or antigen-binding fragment thereof.

In some embodiments, the anti-CD24 antibody or antigen-binding fragment further comprises an Fc region, and the Fc region is selected from mouse IgG1, IgG2a, IgG2b and/or IgG3, or from rat IgG1, IgG2a, IgG2b and/or IgG2c.

In some embodiments, the anti-CD24 antibody or antigen-binding fragment further comprises an Fc region, and the Fc region is selected from human IgG1, IgG2, IgG3 and/or IgG4.

The present invention also provides a nucleic acid molecule encoding any one of the above-described anti-CD24 antibodies or antigen-binding fragments thereof.

The present invention also provides a recombinant vector comprising the above-described nucleic acid molecules.

The present invention also provides a recombinant cell comprising the above-described nucleic acid molecules and/or recombinant vectors and capable of expressing the anti-CD24 antibody or antigen-binding fragment thereof.

The present invention also provides a multifunctional fusion protein comprising any one of the above-described anti-CD24 antibodies or antigen-binding fragments thereof.

In some embodiments, the multifunctional fusion protein further comprises one or more second antibodies or antigen-binding portions thereof that bind specifically to other antigens.

In some embodiments, the antigen binding the second antibody or antigen-binding portion thereof is selected from a tumor-associated antigen (TAA) or an immune checkpoint.

The present invention also provides use of any one of the above-described anti-CD24 antibodies or antigen-binding fragments thereof and the multifunctional fusion proteins in the preparation of a drug for the treatment and/or prevention and/or diagnosis of a disease.

In some embodiments, the use is achieved by one or more of tumor immunotherapy, cell therapy, and gene therapy.

The present invention also provides use of any one of the above-described anti-CD24 antibodies or antigen-binding fragments thereof and the multifunctional fusion proteins in the preparation of a drug for the treatment of cancer.

In some embodiments, the cancer is breast cancer, ovarian cancer, lung cancer, liver cancer, melanoma, malignant glioma, head and neck cancer, colorectal cancer, gastric cancer, bladder cancer, pancreatic cancer, colon cancer, cervical cancer, or a related tumor.

The present invention also provides a pharmaceutical composition comprising any one of the above-described anti-CD24 antibodies or antigen-binding fragments thereof and an acceptable carrier, diluent or excipient.

The present invention also provides a pharmaceutical composition comprising any one of the above-described multifunctional fusion proteins and an acceptable carrier, diluent or excipient.

The anti-CD24 antibodies provided by the present invention have excellent binding activity to MCF7 cells, SKOV3 cells, HT55 cells and huCD24-MDA-MB-231 (breast cancer MDA-MB-231 cells overexpressing CD24). Both the chimeric anti-CD24 antibody and the humanized anti-CD24 antibody provided by the present invention are capable of mediating phagocytosis of MCF7 cells and CD24-SKOV3 cells (SKOV3 cells overexpressing CD24) by macrophages. Moreover, the humanized antibody shows high tumor inhibition in mouse.

To facilitate understanding of the invention set forth herein, the following explanation of abbreviations and definitions of terms are provided.

The following abbreviations are used in this article:
CDR: Complementary determining regions in the variable region of the antibody
HCDR: Complementary determining regions in the variable region of the antibody heavy chain
LCDR: Complementary determining regions in the variable region of the antibody light chain
FR: Antibody construct region, which is the amino acid residues other than CDR residues in the variable region of the antibody
ELISA: Enzyme-linked immunosorbent assay (ELISA)
FACS: Fluorescence Activated Cell Sorting
ADCP: Antibody-dependent cell-mediated phagocytosis

In this specification, the term “antibody” means a natural immunoglobulin or an immunoglobulin prepared by partial or complete synthesis. The antibody may be isolated from a natural source such as plasma or serum in which the antibody is naturally present, or from the culture supernatant of antibody-producing hybridoma cells, or from the immune serum of an animal, or from reconstruction by phage library screening. Alternatively, they may be partially or completely synthesized by using techniques such as genetic recombination. Preferred antibodies include, for example, antibodies to isoforms of immunoglobulins or subclasses of these isoforms. Human immunoglobulins are known to include nine classes (isotypes) of IgG1, IgG2, IgG3, IgG4, IgA1, IgA2, IgD, IgE, and IgM. Within these isotypes, the antibodies of the present invention may include IgG1, IgG2, IgG3, and/or IgG4.

The terms “antibody” and “immunoglobulin” are used interchangeably, and some of the antibodies used herein consist of an immunoglobulin molecule comprising two pairs of polypeptide chains, each pair having a light chain (LC) and a heavy chain (HC). Each heavy chain consists of a heavy chain variable region (VH) and a heavy chain constant region (CH). The heavy chain constant region consists of three structural domains (CH1, CH2 and CH3). Each light chain consists of a light chain variable region (VL) and a light chain constant region (CL), or only a light chain constant region (CL). The light chain constant region consists of a structural domain CL. The constant structural domain is not directly involved in antibody-antigen binding, but exhibits a variety of effector functions, such as can mediate the binding of immunoglobulins to host tissues or factors, including various cells of the immune system (e.g., effector cells) and the first component of the classical complement system (C1q). The VH and VL regions can also be subdivided into regions with high variability (termed complementarity-determining regions (CDRs)), which are scattered with more conserved regions called framing regions (FR). Each VH and VL consists of three CDRs and four FRs arranged from amino-terminal to carboxy-terminal in the following order: FR1, CDR1, FR2, CDR2, FR3, CDR3, FR4. The variable regions of each heavy chain/light chain pair (VH and VL) form the antigen-binding site, respectively.

The term “antigen-binding fragment” of an antibody refers to a polypeptide fragment of an antibody, such as a polypeptide fragment of a full-length antibody, which maintains the ability to specifically bind the same antigen bound by the full-length antibody and/or to compete with the full-length antibody for specific binding of the antigen, and which is also referred to as an “antigen-binding portion”. The antigen-binding fragment of an antibody may be produced by recombinant DNA technology or by enzymatic or chemical breakage of an intact antibody. Non-limiting examples of antigen-binding fragments include Fab, Fab′, F(ab′)2, Fd, Fv, dAb, and complementary determining region (CDR) fragments, single-chain antibodies (e.g., scFv), chimeric antibodies, diabody, linear antibody, nano-antibodies (e.g., technology from Ablynx), structural domain antibodies (e.g., technology from Domantis), and such polypeptides comprising at least a portion of an antibody sufficient to confer specific antigen-binding ability to the polypeptide.

The term “polypeptide” refers to a chain of amino acids of any length, irrespective of modification (e.g. phosphorylation or glycosylation). The term polypeptide includes proteins and fragments thereof. Polypeptides may be “exogenous”, meaning that they are “heterologous”, i.e., foreign to the host cell utilized, such as human polypeptides produced by bacterial cells. Polypeptides are disclosed herein as sequences of amino acid residues. Those sequences are written from left to right in the direction from the amino terminus to the carboxy terminus. According to standard nomenclature, the amino acid residue sequences are named in a three-letter or single-letter code as follows: alanine (Ala, A), arginine (Arg, R), asparagine (Asn, N), aspartate (Asp, D), cysteine (Cys, C), glutamine (Gln, Q), glutamic acid (Glu, E), glycine (Gly, G), histidine (His, H), isoleucine (Ile, I), leucine (Leu, L), lysine (Lys, K), methionine (Met, M), phenylalanine (Phe, F), proline (Pro, P), serine (Ser, S), threonine (Thr, T), tryptophan (Trp, W), tyrosine (Tyr, Y) and valine (Val, V).

There are a variety of methods/systems in the field to define and characterize CDRs that have been developed and refined over the years, including Kabat, Chothia, IMGT, AbM, and Contact; Kabat is the most commonly used and defines CDRs based on sequence variability; Chothia defines CDRs based on sequence variability based on the position of the cyclic region of the structure; the IMGT system defines CDRs based on sequence variability and position within the variable domain structure; AbM is defined based on Oxford Molecules' AbM antibody modeling software and is a compromise between Kabat and Chothia; Contact defines CDRs based on the analysis of complex crystal structures, and is similar to Chothia in several respects.

The amino acid positions are numbered (e.g., amino acid residues in the Fc region) and the target region (e.g., CDR) in the anti-CD24 antibody of the present invention, using the Kabat system.

With respect to the percentage (%) amino acid sequence “identity” of the reference polypeptide sequence is defined as the percentage of amino acid residues in the candidate sequence that are identical to amino acid residues in the reference polypeptide sequence after comparing the sequences and introducing gaps where necessary to obtain the maximum percentage sequence identity. Comparisons for the purpose of determining percent amino acid sequence identity can be performed in a variety of ways within the skill of the art, such as using publicly available computer software, such as BLAST, BLAST-2, Clustal W, Megalign (DNASTAR) software, or the FASTA program package.

The term “monoclonal antibody” refers to a homogeneous antibody directed only to a specific antigenic epitope. In contrast to common polyclonal antibody preparations, which typically include different antibodies directed against different antigenic determinants (epitopes), each monoclonal antibody is directed against a single antigenic determinant on the antigen. The modifier “monoclonal” denotes a homogeneous characterization of the antibody and is not to be construed as requiring that the antibody be produced by any particular method. The monoclonal antibodies of the present invention are preferably produced by recombinant DNA methods or obtained by screening methods described elsewhere herein.

The term “mouse-derived antibody” is used in the present invention to mean a monoclonal antibody prepared according to the knowledge and skill in the art. In some embodiments, the preparation involves injecting a test subject with an antigen and then isolating a hybridoma expressing an antibody having the desired sequence or functional properties. In some embodiments, the desired mouse-derived antibody is obtained by screening a mouse immune library.

The term “chimeric antibody” refers to an antibody that is formed by fusing a variable region of a mouse-derived antibody with a constant region of a human antibody, which can attenuate the immune response induced by the mouse-derived antibody. In some embodiments, to establish a chimeric antibody, a hybridoma secreting a mouse-derived specific monoclonal antibody is established, then the variable region gene is cloned from the mouse hybridoma cells, and then the constant region gene of the human antibody is cloned as needed, and the mouse variable region gene is inserted into a human vector after linking the mouse variable region gene and the human constant region gene to form a chimeric gene, and then finally, the chimeric antibody molecule is expressed in a eukaryotic industrial system or a prokaryotic industrial system.

The term “humanized antibody” means an antibody comprising at least one humanized antibody chain (at least one humanized light or heavy chain). The term “humanized antibody chain” (“humanized immunoglobulin chain”) refers to an antibody chain (light or heavy chain, respectively) having a variable region, the variable region comprising a substantial variable framework region and complementarity determination of the human antibody. Regions (CDRs) substantially derived from a non-human antibody (e.g., at least one CDR, two CDRs, or three CDRs). In some embodiments, the humanized antibody chain further comprises constant regions (e.g., in the case of a light chain, one constant region or a portion thereof, and in the case of a heavy chain, preferably three constant regions).

The term “host cell” means a cell that has been or is capable of being transformed with a nucleic acid sequence and thus expressing the selected target gene. The term includes the progeny of the parental cell, whether or not the progeny is morphologically or genetically identical to the original parental cell, as long as the selected target gene is present in the progeny. Commonly used host cells include bacteria, yeast, mammalian cells, etc.

The term “vector” refers to a nucleic acid molecule capable of proliferating another nucleic acid to which it is attached. The term includes vectors that are self-replicating nucleic acid structures and vectors that are incorporated into the genome of the host cell into which they are introduced. Certain vectors are capable of directing the expression of nucleic acids operably linked to them. Such vectors are referred to herein as “expression vectors”.

### BRIEF DESCRIPTION OF THE DRAWINGS

FIG. 1 shows the binding activity of mouse-derived anti-CD24 antibody to MCF7 cells;

FIG. 2 shows the binding activity of mouse-derived anti-CD24 antibody to SKOV3 cells;

FIGS. 3a-3b show the binding activity of chimeric anti-CD24 antibody to MCF7 cells;

FIGS. 4a-4b show the binding activity of chimeric anti-CD24 antibody to SKOV3 cells;

FIG. 5 shows the binding activity of chimeric anti-CD24 antibody to HT55 cells;

FIGS. 6a-6d show the binding activity of humanized anti-CD24 antibody to MCF7 cells;

FIGS. 7a-7c show the binding activity of humanized anti-CD24 antibody to SKOV3 cells;

FIG. 8 shows the binding activity of humanized anti-CD24 antibody to huCD24-MDA-MB-231 cells;

FIGS. 9a-9b show chimeric and humanized anti-CD24 antibody-mediated ADCP effects on MCF7 cells;

FIGS. 10a-10b show chimeric and humanized anti-CD24 antibody-mediated ADCP effects on CD24-SKOV3 cells.

FIG. 11 shows the in vivo efficacy data of the humanized anti-CD24 antibody.

### DETAILED DESCRIPTION

The present invention is further described below in connection with the accompanying drawings and specific embodiments, the protection of which is not limited to the following embodiments. It should also be understood that the terms used in embodiments of the present invention are intended to describe specific embodiments and are not intended to limit the scope of protection of the present invention. Without departing from the spirit and scope of the inventive concept, variations and advantages that can be envisaged by those skilled in the art are included in the present invention and the scope of protection of the invention by the appended claims and any equivalents thereof. In the specification and claims of the invention, the singular forms “a”, “one” and “this” include the plural forms unless the context expressly states otherwise. The processes, conditions, reagents, experimental methods, etc. for carrying out the invention are, except where specifically mentioned, of general knowledge and common knowledge to those skilled in the art, and the invention is not specifically limited in the present invention.

### Example 1 Animal Immunization

Balb/c mice were immunized with human CD24-his and VLP-huCD24 as immunogens. The immunogen was diluted with PBS, and Fuchs complete adjuvant (Sigma; F5881) was added 1:1 by volume, and vortex emulsified, and the mouse were immunized intraperitoneally after the emulsification was complete. The second immunization was performed half a month after the first immunization, and again every other month after that. Negative serum was taken from mouse 3 days before immunization, and 50 μL of blood was taken from tail clipping 6 days after each immunization. Negative serum and immune serum were diluted proportionally (1:0.1K, 1:1K, 1:10K, 1:100K, 1:1000K, and 1:10,000K), and the breast cancer MDA-MB-231 cells overexpressing CD24 (huCD24-MDA-MB-231) were detected by ELISA for serum potency assay. When the potency result meets the requirement and anti-human CD24 antibody was detected at a dilution of >1:10K, rat spleen and lymph nodes could be harvested.

### Example 2 Cell Fusion

B lymphocytes and lymph node cells for the experiment were obtained from Balb/c mouse immunized four times with recombinant human CD24 protein. The spleens and lymph nodes were placed in a cell sieve, which was then placed in a 50 mL centrifuge tube. DMEM was aspirated and added dropwise to the spleens for grinding to make splenocyte suspension, and centrifuged at 1600 rpm for 10 min, and the supernatant was removed. B cells were resuspended with 2 mL of erythrocyte lysate, and lysed at room temperature for 2 minutes. 30 mL of DMEM was added, mixed and centrifuged at 1600 rpm for 10 min, and counted.

Myeloma cells SP2/0 (ATCC) were passaged the day before fusion so that the cells were in logarithmic growth phase at the time of the experiment. Splenocytes and SP2/0 were mixed at a ratio of 2:1 and then centrifuged at 1600 rpm for 10 min. The mixed cells were washed twice with fusion solution and centrifuged at 1600 rpm for 10 min. According to the final cell density of 1×10⁷, the cells were suspended with fusion solution. Within 5 min, the cell suspensions were transferred to the fusion chamber of the electric fusion apparatus (BTX; ECM 2001) for fusion. After fusion, the cells were transferred from the fusion chamber to complete medium containing HAT and incubated at 37° C. for 60 min. After incubation, the cells were spread in 96-well plates containing feeder cells, and incubated at 37° C. and 5% CO₂.

### Example 3 Preliminary Screening of Positive Clones by ELISA Method

The fusion supernatant was initially screened after 7 days of culture. huCD24-MDA-MB-231 cells were spread into 96 enzyme-labeled plates (1.5×10⁴/per well), incubated for 36 h, and hand-washed with PBST twice. It was fixed with 4% paraformaldehyde and closed with 2% BSA. The sealing solution was poured off and washed with the plate washer 3 times. The fusion supernatant was added into the sealed enzyme-labeled plate at 100 μL/well, and incubated at 37° C. for 1 h, and the liquid in the wells were discarded. It was washed with the plate washer 3 times. Sheep anti-mouse secondary antibody-HRP (ABCOM; Ab6789) was diluted with 0.5% BSA at 100 μL/well and incubated at 37° C. It was washed with PBST in the plate washer 6 times, and patted dry on plate paper. Solarbio color development solution (Solarbio; PR1200) was added into the plate wells at 100 μL/well, and wrapped in aluminum foil, and the color was developed at 37° C. without light. The color development reaction was terminated by adding 1 mol/L M HCl, and the data were read at 450 nm on an enzyme marker, and the data were analyzed. The cell lines with OD450>1.0 in the test supernatant result were selected as candidate positive cell lines for initial screening. The culture supernatant of the positive cell lines was aspirated and discarded, and a new HAT complete medium was added.

### Example 4 FACS Method for Further Screening of Positive Clones

huCD24-MDA-MB-231 cells were transferred to a centrifuge tube and centrifuged at 1000 rpm for 5 min. 100 μL of 3×10⁵ stably expressing cells were aliquoted into separate tubes and 100 μL of fusion supernatant was added. The cells were incubated at 4° C. for 60 minutes and then washed twice with excess FACS buffer. Cells were resuspended in 100 μL of FACS buffer, and sheep anti-mouse secondary antibody-FITC (ABCOM; ab6785) was added to the sample, incubated for 30 minutes and washed twice with excess FACS buffer. Cells were fixed in fixation buffer and subsequently analyzed by flow cytometry. The antibodies that bind specifically to huCD24-MDA-MB-231 cells were screened by FACS method.

The hybridoma cells were monoclonalized by two rounds of limited dilution method, and ELISA was used for detection. The monoclones with OD450>1.0 were selected as the definitive candidate cell lines for passage, and clones without monoclonal antibodies were selected with OD450>1.0 for the next subcloning.

### Example 5 Small Sample Production of Antibodies to Candidate Cell Lines

The hybridoma cells were incubated in T75 until the cell coverage was 80-90%. The cell supernatant was discarded from 2 bottles, and 30 mL hybridoma-SFM was added, incubated at 37° C. and 5% CO₂ for 2-3 days, and observed the cell status and medium color. If the medium color turned yellow, 30 mL of new hybridoma-SFM was added and incubated for 6-7 days, and the culture supernatant was collected after low-speed centrifugation for purification.

### Example 6 Candidate Antibody Binding Activity to Tumor Cells

The final positive clones were confirmed by evaluating the binding activity of the candidate antibodies to tumor cells MCF7 (ATCC), SKOV3 (ATCC), HT55 (ATCC), and huCD24-MDA-MB-231 using the FACS method. The commercial antibody SN3 (ABCOM; ab134375) and IgG1 isotype control were used as control. MCF7, SKOV3, HT55 or huCD24-MDA-MB-231 cells were taken and transferred to a centrifuge tube, and centrifuged at 1000 rpm for 5 min. 100 μL of MCF7, SKOV3, HT55, or huCD24-MDA-MB-231 cells were spread in a 96 U plate, respectively, and washed twice with PBS. 100 μL of the fusion supernatant was added, and diluted for 8 gradients. Cells were incubated at 4° C. for 60 minutes, and then washed twice with excess FACS buffer. Cells were resuspended in 100 μL of FACS buffer and sheep anti-mouse secondary antibody-FITC (ABCOM; ab6785) was added to the samples, incubated for 30 minutes and washed twice with excess FACS buffer. Cells were fixed in fixation buffer and subsequently analyzed by flow cytometry.

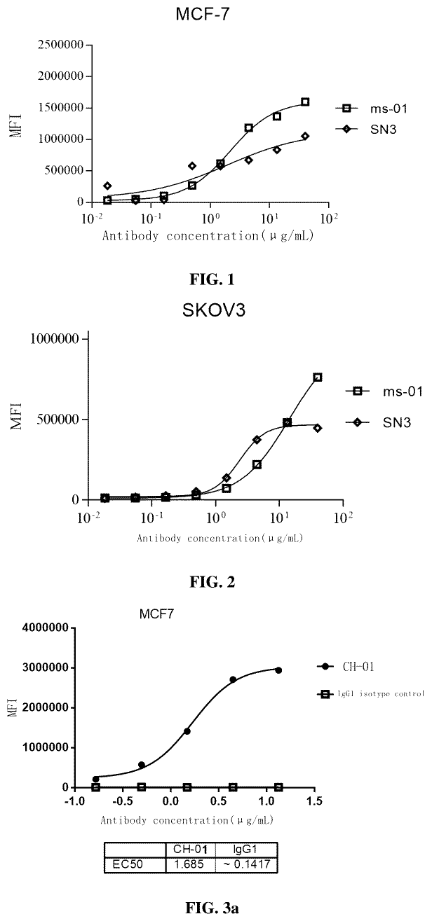

The binding activity of ms-01 and MCF7 cells is shown in FIG. 1. As can be seen from the results, the binding activity of ms-01 and MCF7 cells is better than the control antibody SN3 or comparable to that of the control antibody SN3.

The binding activity of ms-01 and SKOV3 cells is shown in FIG. 2. As can be seen from the results, the binding activity of ms-01 and SKOV3 cells is better than or comparable to that of the control antibody SN3.

### Example 7 Construction of the Mouse Immunization Library

The spleen of immunized mouse was taken, and RNA was extracted from the mouse spleen tissue by chloroform method. After the RNA was identified and the concentration was determined to be of satisfactory quality, the RNA was reverse transcribed according to the method provided by the TaKaRa Reverse Transcription Kit. The immunized mouse antibody gene was amplified by universal concatenated primers, and then the antibody gene was recombined by in vitro ligation, and finally the recombined antibody Fab gene sequence was inserted into PMID-1121B vector, thus the mouse immunized antibody library was obtained. In addition, the library capacity of the antibody library was determined by the dilution spot plate method, and the correct insertion rate of the antibody gene was verified by monoclonal sequencing analysis. The results showed that the library capacity of the antibody library was larger than the designed capacity, and the correct insertion rate of the antibody library was more than 80%, which could reflect the high abundance from CDR3, and the quality of this library construction was qualified and could be used for antibody screening.

### Example 8 Mouse Immune Bank Screening

With Human CD24-his and VLP-huCD24 as screening proteins, Kingfisher method or the immunotube method were used to select in the constructed mouse immune library. Then huCD24-HEK293, huCD24-MDA-MB-231 and huCD24-SKOV-3 were used as screening cells for cell panning of the constructed mouse immune library, and finally positive antibodies were screened by ELISA method. The specific steps were as follows:

### 1. Kingfisher Method or the Immunotube Method

Elution: Phage was eluted with Trypsin.

Immersion library plate: The eluted phage solution was mixed thoroughly with log phase SS320 cells and incubated statically. It was coated on 2YT-C+-T+ plate, and cultured overnight in 37° C. incubator.

Spot Titer: After elution, the phage solution was diluted with logarithmic SS320 cells in 10-fold gradient and incubated statically. After mixing well, 2 μL was spotted on the plate and incubated in an incubator at 37° C. overnight.

Statistics: Phage output, input, etc. were calculated, and input phage was prepared by scraping plate, and subsequent screening was carried out. Each round of screening would reduce the concentration of the antigen by 3 times to ensure that Fab with good affinity was screened.

### 2. Cell Panning Using Live and Fixed Cells Respectively

(1) Live cell panning: According to the number of rounds of panning and the requirements of cell screening, the cells were pre-distributed into 15 mL centrifuge tubes according to the amount and kept at a low temperature. The closed phage was added into the treated cells. Before that, it is necessary that the closed phage was added into the negative screening cells, mixed, combined, and centrifuged. The supernatant of the phage library after negative screening was added to the corresponding positive screening cells, mixed, combined and centrifuged. The supernatant was discarded and washed with pre-cooled 5% FBS-PBS. Phage library elution was then performed.

(2) Fixed cell panning: the closed phage was absorbed with a disposable pipette and slowly added to the treated cell bottle on the side wall. Before that, negative screening was required. The closed phage was first added into the negative screening cells and placed on the track-type shaker for mixing and combining for 1 h. After combination, the cell culture bottle was gently stood up, and the phage library after negative screening was sucked out and added to the corresponding positive screening cell bottle using a disposable pipette. It was mixed and combined on the track-type shaker at room temperature for 2 h. The supernatant in the culture bottle was discarded, and 5% FBS-PBS was added. Then the cell plate was placed in the upright position, and carefully suck out with a disposable pipette to complete the washing. Phage library elution was then performed.

### 3. Preliminary Screening of Monoclones by ELISA

The second and third rounds of clones were selected and cultured overnight in 96-well deep-well plates. The supernatant was centrifuged for ELISA screening. 30 μL of antigen-coated synthetic CD24 extracellular region peptide, huCD24-his and VLP-hCD24 at 2 μg/mL were added to each well at 4° C. overnight. The plate was washed 3 times with PBST. 5% PBSM was added and blocked at room temperature for 1 h. The plate was washed 3 times with PBST. Monoclones were added at 30 μL/well at room temperature for 1 h, and the plate was washed 3 times with PBST. 30 μL of secondary antibody Anti-M13-HRP (1:8000) was added at room temperature for 1 h, and the plate was washed 9 times with PBST. 30 μL of TMB was added to develop the color at room temperature for 5-10 min, after which the reaction was terminated by adding 30 μL of 2 M HCl. The data were read by the enzyme marker at OD450.

### 4. Quantification of Phage Lysate by ELISA

Anti-mouse-Fab was diluted with PBS to 1 μg/mL, added at 30 μL per well and left to stand at 4° C. overnight. Plates were washed 3 times with PBST. It was blocked with 5% PBSM at room temperature for 1 h, and the plate was washed 3 times with PBST. 30 μL of gradient diluted phage lysate was added at room temperature for 1 h, and the plate was washed 3 times with PBST. 30 μL of diluted secondary antibody Anti-mouse-Fab-HRP (1:5000) was added at room temperature for 1 h, and the plate was washed 9 times with PBST. 30 μL of TMB was added to develop the color at room temperature for 5-20 min, after which the reaction was terminated by adding 30 μL of 2 mol/L HCl. The data were read by the enzyme marker at OD450.

### Example 9 Sequencing of Monoclonal Antibodies

The murine anti-CD24 antibody ms-01 with good binding activity to tumor cells MCF7 and SKOV3 was obtained by hybridoma screening, and Fab fragment of the murine anti-CD24 antibody ms-02 was obtained by mouse immune library screening. The amino acid sequences of murine anti-CD24 antibody ms-01 and ms-02 were finally obtained by sequencing ms-01 and ms-02, respectively:

(1) The amino acid sequences of ms-01 are as follows:
The amino acid sequence of the heavy chain variable region of ms-01 is SEQ ID NO: 1;
The amino acid sequences of HCDR1, HCDR2 and HCDR3 of the heavy chain variable region of ms-01 are SEQ ID NO: 2, SEQ ID NO: 3 and SEQ ID NO: 4, respectively;
The amino acid sequence of the light chain variable region of ms-01 is SEQ ID NO: 5;
The amino acid sequences of LCDR1, LCDR2 and LCDR3 of the light chain variable region of ms-01 are SEQ ID NO: 6, SEQ ID NO: 7 and SEQ ID NO: 8, respectively.

(2) The amino acid sequences of ms-02 are as follows:
The amino acid sequence of the heavy chain variable region of ms-02 is SEQ ID NO: 16;
The amino acid sequences of HCDR1, HCDR2 and HCDR3 of the heavy chain variable region of ms-02 are SEQ ID NO: 17, SEQ ID NO: 18 and SEQ ID NO: 19, respectively;
The amino acid sequence of the light chain variable region of ms-02 is SEQ ID NO: 20;
The amino acid sequences of LCDR1, LCDR2 and LCDR3 of the light chain variable region of ms-02 are SEQ ID NO: 21, SEQ ID NO: 22 and SEQ ID NO: 23, respectively.

### Example 10 Construction and Expression of Chimeric Antibodies

The antibody fragment obtained by sequencing in Example 9 was subjected to gene synthesis and constructed into a human IgG framework, and then the antibody fragment was inserted into a PCDNA3.1 vector using molecular cloning technology, constructed into a mammalian cell expression plasmid, and introduced into a host cell line CHO cell by liposome transfection. The fermentation supernatant was obtained by cell fed-batch. The supernatant of the fermentation fluid was taken for purification by affinity chromatography, ion exchange chromatography and a series of other steps, and the constructed chimeric antibody was finally purified.

(1) The amino acid sequences of CH-01 are as follows:
The amino acid sequence of the heavy chain variable region of CH-01 is SEQ ID NO: 1;
The amino acid sequences of HCDR1, HCDR2 and HCDR3 of the heavy chain variable region of CH-01 are SEQ ID NO: 2, SEQ ID NO: 3 and SEQ ID NO: 4, respectively;
The amino acid sequence of the heavy chain constant region of CH-01 is SEQ ID NO: 35;
The amino acid sequence of the light chain variable region of CH-01 is SEQ ID NO: 5;
The amino acid sequences of LCDR1, LCDR2 and LCDR3 of the light chain variable region of CH-01 are SEQ ID NO: 6, SEQ ID NO: 7 and SEQ ID NO: 8, respectively;
The amino acid sequence of the light chain constant region of CH-01 is SEQ ID NO: 36.

(2) The amino acid sequences of CH-02 are as follows:
The amino acid sequence of the heavy chain variable region of CH-02 is SEQ ID NO: 16;
The amino acid sequences of HCDR1, HCDR2 and HCDR3 of the heavy chain variable region of CH-02 are SEQ ID NO: 17, SEQ ID NO: 18 and SEQ ID NO: 19, respectively;
The amino acid sequence of the heavy chain constant region of CH-02 is SEQ ID NO: 35;
The amino acid sequence of the light chain variable region of CH-02 is SEQ ID NO: 20;
The amino acid sequences of LCDR1, LCDR2 and LCDR3 of the light chain variable region of CH-02 are SEQ ID NO: 21, SEQ ID NO: 22 and SEQ ID NO: 23, respectively;
The amino acid sequence of the light chain constant region of CH-02 is SEQ ID NO: 36.

### Example 11 Binding Activity of Chimeric Antibodies to Tumor Cells

The binding activity of the chimeric antibodies CH-01 and CH-02 and tumor cells was detected according to the method of Example 6.

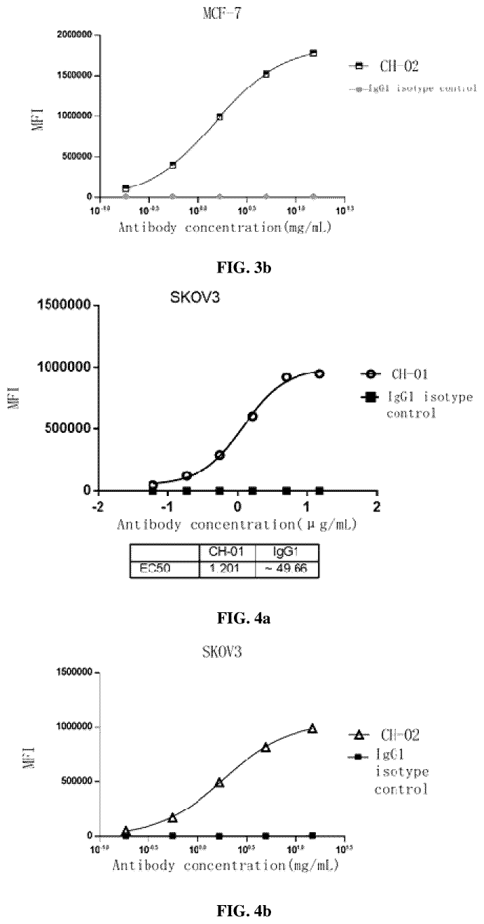

The binding activity of the chimeric antibodies and MCF7 cells is shown in FIGS. 3a-3b. As can be seen from the results, the binding activity of CH-01 and CH-02 and MCF7 cells was significantly better than that of the IgG1 isotype control.

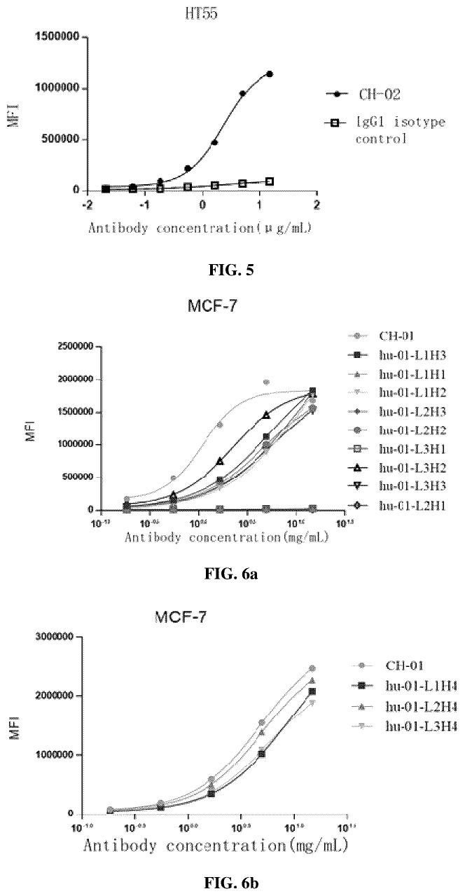

The binding activity of the chimeric antibodies and SKOV3 cells is shown in FIGS. 4a-4b. As can be seen from the results, the binding activity of CH-01 and CH-02 and SKOV3 cells was significantly better than that of the IgG1 isotype control.

The binding activity of the chimeric antibodies and HT55 cells is shown in FIG. 5. As can be seen from the results, the binding activity of CH-02 and HT55 cells was significantly better than that of the IgG1 isotype control.

### Example 12 Antibody Humanization

The IgBLAS tool was used to align the murine sequence obtained in Example 9 with the human Germline sequence. The results showed that the heavy chain variable region Framework 1-3 contained 18 murine sites (V genes), and the light chain variable region Framework 1-3 contained 23 murine sites (V genes). The heavy chain design template selected the IGHV7 category, and the light chain design template selected the IGHV1 category. Humanized sequences were designed and the sequences are mutated into humanized sequences. 4 heavy chains and 3 light chains of the humanized antibody were obtained from the ms-01, and 8 heavy chains and 3 light chains of the humanized antibody were obtained from the ms-02. The designed humanized sequences were compared with the human Germline sequences and the percentage of humanization degree of the antibodies was calculated to be above 80%.

(1) Four humanized antibody heavy chains H1, H2, H3 and H4 with all three identical HCDRs were obtained from ms-01, and the amino acid sequences of HCDR1, HCDR2 and HCDR3 in the variable region of the heavy chains were SEQ ID NO: 2, SEQ ID NO: 3 and SEQ ID NO: 4, respectively; and three humanized antibody light chains L1, L2 and L3 were obtained which were identical to the three LCDRs, and the amino acid sequences of HCDR1, HCDR2 and HCDR3 in the variable regions of their light chains were SEQ ID NO: 6, SEQ ID NO: 7 and SEQ ID NO: 8, respectively.

The amino acid sequences of the variable regions of the heavy chains H1, H2, H3 and H4 of the four humanized antibodies are shown in Table 1:

TABLE 1

| Heavy chain | Variable region amino acid |
|---|---|
| numbers | sequences |
| H1 | SEQ ID NO: 9 |
| H2 | SEQ ID NO: 10 |
| H3 | SEQ ID NO: 11 |
| H4 | SEQ ID NO: 12 |

The constant regions of the heavy chains H1, H2, H3 and H4 of the four humanized antibodies are identical, and the amino acid sequence of their constant regions is SEQ ID NO: 35.

The amino acid sequences of the variable regions of the light chains L1, L2 and L3 of the three humanized antibodies are shown in Table 2:

TABLE 2

| Light chain | Variable region amino acid |
|---|---|
| numbers | sequences |
| L1 | SEQ ID NO: 13 |
| L2 | SEQ ID NO: 14 |
| L3 | SEQ ID NO: 15 |

The constant regions of the three humanized antibody light chains L1, L2 and L3 are identical, and the amino acid sequence of their constant regions is SEQ ID NO: 36.

The above four heavy chains and three light chains were grouped into 12 humanized antibodies, namely: hu-01-L1H1, hu-01-L2H1, hu-01-L3H1, hu-01-L1H2, hu-01-L2H2, hu-01-L3H2, hu-01-L1H3, hu-01-L2H3, hu-01-L3H3, hu-01-L1H4, hu-01-L2H4, and hu-01-L3H4, respectively.

(2) Eight humanized antibodies H1, H2, H3, H4, H5, H6, H7 and H8 with all three identical HCDRs were obtained from ms-02, and the amino acid sequences of HCDR1, HCDR2 and HCDR3 in the variable region of the heavy chain were SEQ ID NO: 17, SEQ ID NO: 18 and SEQ ID NO: 19, respectively; and three humanized antibody light chains L1, L2, and L3 with the same LCDR were obtained. The amino acid sequences of HCDR1, HCDR2 and HCDR3 in the variable regions of the light chains of the three humanized antibodies were SEQ ID NO: 21, SEQ ID NO: 22 and SEQ ID NO: 23, respectively.

The amino acid sequences of the variable regions of the heavy chains H1, H2, H3, H4, H5, H6, H7 and H8 of the eight humanized antibodies are shown in Table 3:

TABLE 3

| Heavy chain | Variable region amino acid |
|---|---|
| numbers | sequences |
| H1 | SEQ ID NO: 24 |
| H2 | SEQ ID NO: 25 |
| H3 | SEQ ID NO: 26 |
| H4 | SEQ ID NO: 27 |
| H5 | SEQ ID NO: 28 |
| H6 | SEQ ID NO: 29 |
| H7 | SEQ ID NO: 30 |
| H8 | SEQ ID NO: 31 |

The constant regions of the heavy chains H1, H2, H3, H4, H5, H6, H7 and H8 of the eight humanized antibodies are identical, and the amino acid sequences of their constant regions is SEQ ID NO: 35.

The amino acid sequences of the variable regions of the light chains L1, L2 and L3 of the three humanized antibodies are shown in Table 4:

TABLE 4

| Light chain | Variable region amino acid |
|---|---|
| numbers | sequences |
| L1 | SEQ ID NO: 32 |
| L2 | SEQ ID NO: 33 |
| L3 | SEQ ID NO: 34 |

The constant regions of the three humanized antibody light chains L1, L2 and L3 are identical, and the amino acid sequence of their constant regions is SEQ ID NO: 36.

The above eight heavy chains and three light chains were grouped into 24 humanized antibodies, namely: hu-02-L1H1, hu-02-L2H1, hu-02-L3H1, hu-02-L1H2, hu-02-L2H2, hu-02-L3H2, hu-02-L1H3, hu-02-L2H3, hu-02-L3H3, hu-02-L3H3, hu-02-L1H4, hu-02-L2H4, hu-02-L3H4, hu-02-L1H5, hu-02-L2H5, hu-02-L3H5, hu-02-L1H6, hu-02-L2H6, hu-02-L3H6, hu-02-L1H7, hu-02-L2H7, hu-02-L3H7, hu-02-L1H8, hu-02-L2H8, and hu-02-L3H8.

### Example 13 Affinity Assay for Humanized Antibodies

ForteBio Biofilm Interference (BLI) was used to detect the affinity of humanized antibodies. The sample to be tested (solidified) was diluted to 20 μg/mL with 0.02% PBST (buffer). The analyte human CD24 protein (ACRO) was diluted to 100 nM with 0.02% PBST (buffer) as the initial concentration, and then diluted by double gradient for 7 gradients starting from this concentration, and a negative control (buffer) was performed.

The molecular interaction analysis instrument (ForteBio; Octet RED96e) was operated, and 200 μL of curing buffer (0.02% PBST) was added to a pre-wetted plate, and the sensor was pre-wetted for 5-10 min. Diluted samples and other reagents for assay were added into wells in the sample plate corresponding to the pre-wetted sensor.

Through the analysis of the experimental data, the affinity assay data for the humanized antibodies were obtained as shown in Table 5 and Table 6.

TABLE 5

| Antibody Name | K_{D} (mol/L) |
|---|---|
| CH-01 | 8.08 × 10⁻⁰⁹ |
| hu-01-L1H1 | No bonding |
| hu-01-L1H2 | 5.58 × 10⁻⁰⁹ |
| hu-01-L1H3 | 1.00 × 10⁻⁰⁸ |
| hu-01-L1H4 | 4.82 × 10⁻⁰⁸ |
| hu-01-L2H1 | No bonding |
| hu-01-L2H2 | 1.05 × 10⁻⁰⁸ |
| hu-01-L2H3 | 9.03 × 10⁻⁰⁹ |
| hu-01-L2H4 | 3.91 × 10⁻⁰⁸ |
| hu-01-L3H1 | No bonding |
| hu-01-L3H2 | 8.29 × 10⁻⁰⁹ |
| hu-01-L3H3 | 1.24 × 10⁻⁰⁸ |
| hu-01-L3H4 | 4.12 × 10⁻⁰⁸ |

TABLE 6

| Antibody Name | K_{D} (mol/L) |
|---|---|
| CH-02 | 1.19 × 10⁻⁰⁸ |
| hu-02-L1H1 | 6.27 × 10⁻⁰⁸ |
| hu-02-L2H1 | 3.27 × 10⁻⁰⁸ |
| hu-02-L3H1 | 4.47 × 10⁻⁰⁸ |
| hu-02-L1H2 | Untested |
| hu-02-L2H2 | 3.58 × 10⁻⁰⁸ |
| hu-02-L3H2 | 2.27 × 10⁻⁰⁸ |
| hu-02-L1H7 | 3.00 × 10⁻⁰⁸ |
| hu-02-L2H3 | 3.25 × 10⁻⁰⁸ |
| hu-02-L3H3 | 4.35 × 10⁻⁰⁸ |
| hu-02-L1H4 | Untested |
| hu-02-L2H4 | 2.79 × 10⁻⁰⁸ |
| hu-02-L3H4 | 3.26 × 10⁻⁰⁸ |
| hu-02-L1H5 | Untested |
| hu-02-L2H5 | 2.68 × 10−⁰⁸ |
| hu-02-L3H5 | 2.45 × 10⁻⁰⁸ |
| hu-02-L1H6 | Untested |
| hu-02-L2H6 | 2.58 × 10⁻⁰⁸ |
| hu-02-L3H6 | 2.95 × 10⁻⁰⁸ |
| hu-02-L1H7 | 2.05 × 10⁻⁰⁸ |
| hu-02-L2H7 | 1.23 × 10⁻⁰⁸ |
| hu-02-L3H7 | 1.35 × 10⁻⁰⁸ |
| hu-02-L1H8 | Untested |
| hu-02-L2H8 | 8.23 × 10⁻⁰⁹ |
| hu-02-L3H8 | 1.00 × 10⁻⁰⁸ |

### Example 14 Binding Activity of Humanized Antibodies to Tumor Cells

The binding activity of the humanized antibodies to the tumor cells was detected according to the method of Example 8.

The binding activity of the humanized antibodies and MCF7 cells is shown in FIGS. 6a-6d. As can be seen from the results, except for hu-01-L1H1 and hu-01-L3H1, which have poor binding activity and MCF7 cells, all other humanized antibodies have good binding activity and MCF7 cells, which are comparable to the chimeric antibodies.

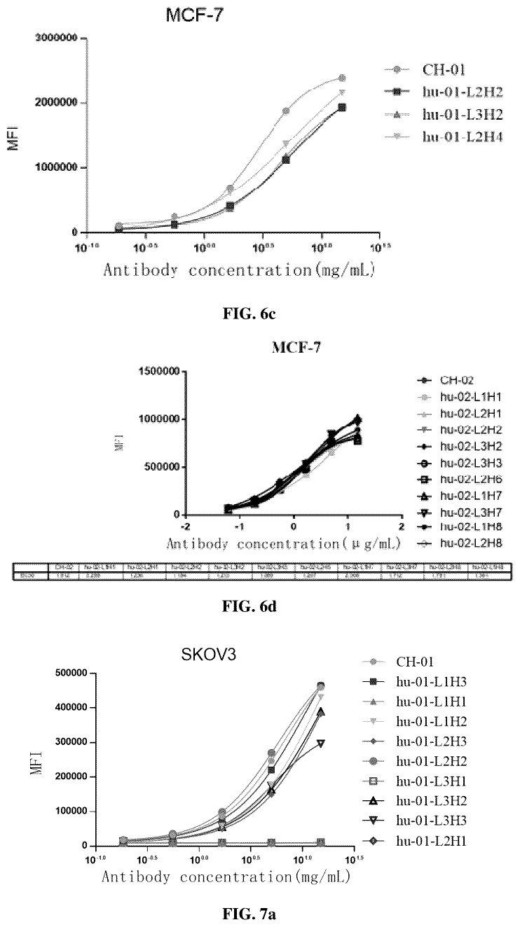

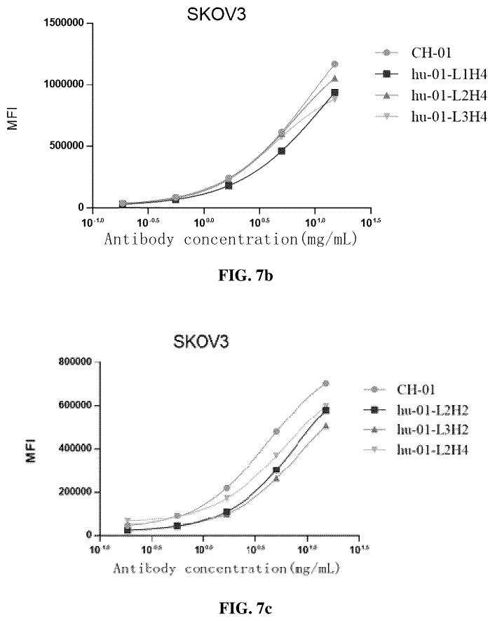

The binding activity of the humanized antibodies and SKOV3 cells is shown in FIGS. 7a-7c. As can be seen from the results, except for hu-01-L1H1 and hu-01-L3H1, which have poor binding activity and SKOV3 cells, all other humanized antibodies have good binding activity and MCF7 cells, which are comparable to the chimeric antibodies.

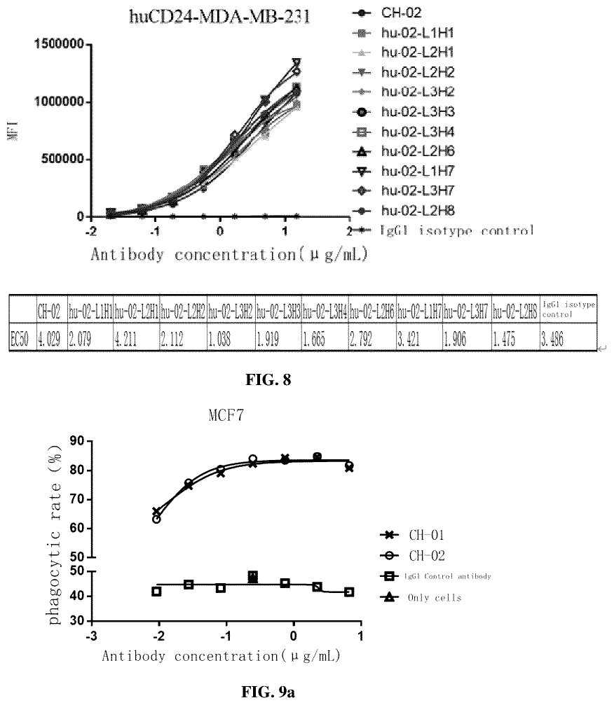

The binding activity of the humanized antibodies and huCD24-MDA-MB-231 cells is shown in FIG. 8. As can be seen from the results, the binding activity of the humanized anti-CD24 antibodies and huCD24-MDA-MB-231 cells are significantly better than those of the chimeric antibody CH-02 and the control antibody.

### Example 15 ADCP Effects Mediated by Chimeric and Humanized Antibodies

Antibodies were diluted with FACS buffer into diluents with different concentration gradients. Antibody diluents were added into the 96-well round-bottom plate using a 100 μL pipette displacement gun at 50 μL per well.

After the cells were evenly dispersed and counted, the cells were washed with PBS. The cell density of tumor cells (MCF7 cells or SKOV3 cells) was adjusted to 1×10⁶ cells/mL with pre-warmed PBS, and CFSE was added to make its concentration to 1 μM. The cells were incubated at 37° C. for 20 min, and centrifuged to remove the supernatant. The cells were resuspended by adding 5 mL of 1640+10% FBS complete medium. The cells were centrifuged after the incubation was completed, and the cells were resuspended with 1640+10% FBS complete medium and counted The cell density was adjusted to 2×10⁶ cells/mL for use. The diluted cells were poured into a sterile sampling tank, and the cells were added to the above cell plates using a 300 μL 12-channel discharge gun at 50 μL per well, and the number of tumor cells per well was 1×10⁵/well.

The monocytes were induced into macrophages, and cells were digested by accutase. Macrophages were made into cell suspension, and centrifuged, and supernatant was discarded. RPMI1640 complete medium was added for resuspension counting, and the cell density was adjusted to 1×10⁶ cells/mL. Antibody diluent was added into the 96-well round-bottomed plate with a 100 μL pipette volley gun, each well was 100 μL, and gently blew a few times. The two cells and antibody diluent were mixed, and co-incubated at 37° C. for 3 h.

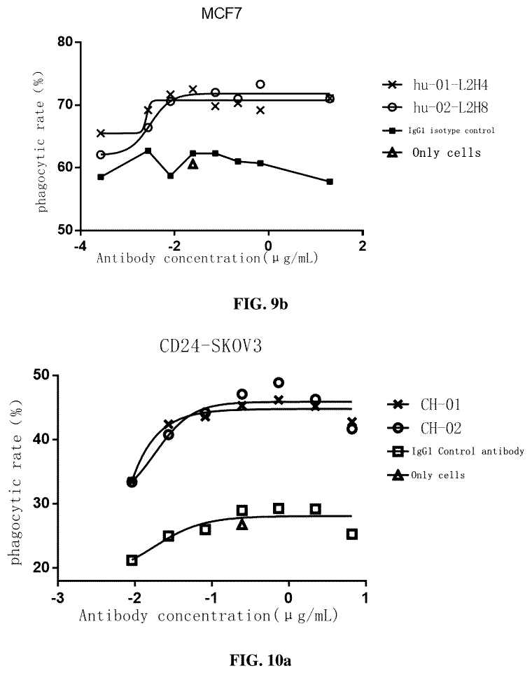

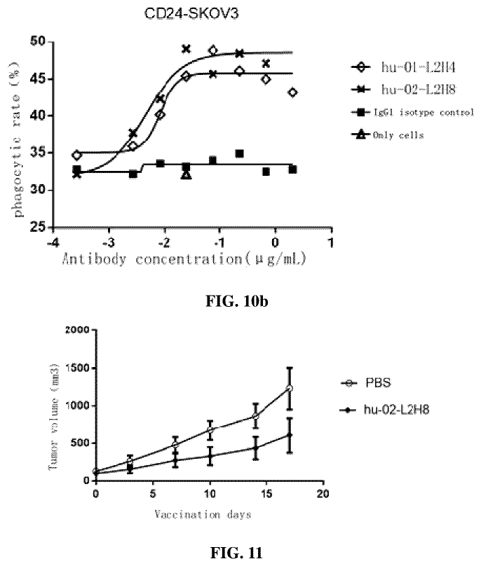

Adding the secondary antibody: the incubated 96-well round-bottomed plate was put into the centrifuge and centrifuged. The supernatant was discarded. The secondary antibody CD14 Monoclonal Antibody (61D3) APC was diluted at 1:100 with FACS buffer, and it was added to each well at 100 μL/well, and incubated at 4° C. for 20 min. The plate was washed twice with FACS buffer. The samples were detected and analyzed. The results are shown in FIGS. 9a-9b and FIGS. 10a-10b.

As can be seen from the results, CH-01, CH-02, hu-01-L2H4 and hu-02-L2H8 all mediate the phagocytosis of MCF7 cells and CD24-SKOV3 cells by macrophages.

### Example 16 in Vivo Pharmacodynamic Experiments with Humanized Antibodies

A subcutaneous tumor model of ovarian cancer SKOV3 cells in BALB/C nude mice was established to evaluate in vivo anti-tumor efficacy of the humanized antibody hu-02-L2H8. SKOV3 cells were resuscitated, cell culture was performed, and cell suspension was prepared after digestion. When the cells were cultured to the logarithmic growth phase, the cells were collected, and the tumor cell suspension was injected into the subcutaneous of BALB/C nude mice, each of which was inoculated with 100 μL of cell suspension containing 5×10⁶ cells. Tumor growth was observed and tumor growth curves were plotted.

When the subcutaneous tumor grew to about 500 mm³, the subcutaneous tumor was stripped, divided into 1 mm³ tumor tissue blocks in the medium, and transplanted to the subcutaneous back of BALB/C nude mice. When the average volume of subcutaneous tumors reached 75-125 mm³, the animals were randomly grouped according to tumor volume and given anti-CD24 antibody treatment.

The grouped tumor-bearing mice were given intraperitoneal injections of the antibodies hu-02-L2H8 and PBS, respectively, and 10.0 mg/kg was administered twice a week for a total of five times.

The tumor-suppressive efficacy of the compounds was evaluated by TGI (%). The formula for calculating TGI (%) was: TGI (%)=[1−(average tumor volume at the end of administration of a treatment group−average tumor volume at the beginning of administration of this treatment group)/(average tumor volume at the end of treatment of solvent control group−average tumor volume at the beginning of treatment of solvent control group)]×100%.

The tumor growth curves of the mouse ovarian cancer SKOV3 cell subcutaneous tumor model of hormonal mice given the antibody are described in FIG. 8, where the number of days after the start of treatment was indicated by the horizontal coordinate and the tumor volume was indicated by the vertical coordinate. The tumor suppression rate TGI (%) was greater than 50%.

As shown in FIG. 11, the humanized antibody hu-02-L2H8 had a high tumor inhibitory effect, which was significantly better than that of the PBS group.

1. (canceled)

2. An anti-CD24 antibody or antigen-binding fragment thereof, comprising a heavy chain variable region and a light chain variable region, wherein the heavy chain variable region comprises the heavy chain complementary determining regions HCDR1, HCDR2, and HCDR3, and the light chain variable region comprises the light chain complementary determining regions LCDR1, LCDR2, and LCDR3, wherein
(a) HCDR1 of the heavy chain variable region is selected from any amino acid sequence of SEQ ID NOs: 2, 17, or a sequence having at least 80%, 85%, 90%, 91%, 92%, 93%, 94%, 95%, 96%, 97%, 98%, 99% or more identity to any amino acid sequence of SEQ ID NOs: 2, 17, or an amino acid sequence having one or more conserved amino acid mutations in comparison to any amino acid sequence of SEQ ID NOs: 2, 17;
(b) HCDR2 of the heavy chain variable region is selected from any amino acid sequence of SEQ ID NOs: 3, 18, or a sequence having at least 80%, 85%, 90%, 91%, 92%, 93%, 94%, 95%, 96%, 97%, 98%, 99% or more identity to any amino acid sequence of SEQ ID NOs: 3, 18, or an amino acid sequence having one or more conserved amino acid mutations in comparison to any amino acid sequence of SEQ ID NOs: 3, 18;
(c) HCDR3 of the heavy chain variable region is selected from any amino acid sequence of SEQ ID NOs: 4, 19, or a sequence having at least 80%, 85%, 90%, 91%, 92%, 93%, 94%, 95%, 96%, 97%, 98%, 99% or more identity to any amino acid sequence of SEQ ID NOs: 4, 19, or an amino acid sequence having one or more conserved amino acid mutations in comparison to any amino acid sequence of SEQ ID NOs: 4, 19;
(d) LCDR1 of the light chain variable region is selected from any amino acid sequence of SEQ ID NOs: 6, 21, or a sequence having at least 80%, 85%, 90%, 91%, 92%, 93%, 94%, 95%, 96%, 97%, 98%, 99% or more identity to any amino acid sequence of SEQ ID NOs: 6, 21, or an amino acid sequence having one or more conserved amino acid mutations in comparison to any amino acid sequence of SEQ ID NOs: 6, 21;
(e) LCDR2 of the light chain variable region is selected from any amino acid sequence of SEQ ID NOs: 7, 22, or a sequence having at least 80%, 85%, 90%, 91%, 92%, 93%, 94%, 95%, 96%, 97%, 98%, 99% or more identity to any amino acid sequence of SEQ ID NOs: 7, 22, or an amino acid sequence having one or more conserved amino acid mutations in comparison to any amino acid sequence of SEQ ID NOs: 7, 22; and/or
(f) LCDR3 of the light chain variable region is selected from any amino acid sequence of SEQ ID NOs: 8, 23, or a sequence having at least 80%, 85%, 90%, 91%, 92%, 93%, 94%, 95%, 96%, 97%, 98%, 99% or more identity to any amino acid sequence of SEQ ID NOs: 8, 23, or an amino acid sequence having one or more conserved amino acid mutations in comparison to any amino acid sequence of SEQ ID NOs: 8, 23.

3. The anti-CD24 antibody or antigen-binding fragment thereof according to claim 2, wherein HCDR1, HCDR2, and HCDR3 of the heavy chain variable region consist of the amino acid sequences SEQ ID NO: 2, SEQ ID NO: 3, and SEQ ID NO: 4, respectively, and LCDR1, LCDR2, and LCDR3 of the light chain variable region consist of SEQ ID NO: 6, SEQ ID NO: 7, and SEQ ID NO: 8, respectively.

4. The anti-CD24 antibody or antigen-binding fragment thereof according to claim 2, wherein the heavy chain variable region has the amino acid sequence given in SEQ ID NO: 1 and the light chain variable region that has the amino acid sequence given in SEQ ID NO: 5.

5. The anti-CD24 antibody or antigen-binding fragment thereof according to claim 2, wherein the heavy chain variable region and light chain variable region are selected from a group consisting of the following (1)-(12) amino acid sequence combinations:

| NO. | Heavy chain variable region | Light chain variable region |
|---|---|---|
| (1) | SEQ ID NO: 9 | SEQ ID NO: 13 |
| (2) | SEQ ID NO: 9 | SEQ ID NO: 14 |
| (3) | SEQ ID NO: 9 | SEQ ID NO: 15 |
| (4) | SEQ ID NO: 10 | SEQ ID NO: 13 |
| (5) | SEQ ID NO: 10 | SEQ ID NO: 14 |
| (6) | SEQ ID NO: 10 | SEQ ID NO: 15 |
| (7) | SEQ ID NO: 11 | SEQ ID NO: 13 |
| (8) | SEQ ID NO: 11 | SEQ ID NO: 14 |
| (9) | SEQ ID NO: 11 | SEQ ID NO: 15 |
| (10) | SEQ ID NO: 12 | SEQ ID NO: 13 |
| (11) | SEQ ID NO: 12 | SEQ ID NO: 14 |
| (12) | SEQ ID NO: 12 | SEQ ID NO: 15. |

6. The anti-CD24 antibody or antigen-binding fragment thereof according to claim 2, wherein HCDR1, HCDR2, and HCDR3 of the heavy chain variable region consist of the amino acid sequences SEQ ID NO: 17, SEQ ID NO: 18, and SEQ ID NO: 19, respectively, and LCDR1, LCDR2, and LCDR3 of the light chain variable region consist of SEQ ID NO: 21, SEQ ID NO: 22, and SEQ ID NO: 23, respectively.

7. The anti-CD24 antibody or antigen-binding fragment thereof according to claim 5, wherein the heavy chain variable region has the amino acid sequence given in SEQ ID NO: 16 and the light chain variable region has the amino acid sequence given in SEQ ID NO: 20.

8. The anti-CD24 antibody or antigen-binding fragment thereof according to claim 56, wherein the heavy chain variable region and light chain variable region are selected from a group consisting of the following (1)-(24) amino acid sequence combinations:

| NO. | Heavy chain variable region | Light chain variable region |
|---|---|---|
| (1) | SEQ ID NO: 24 | SEQ ID NO: 32 |
| (2) | SEQ ID NO: 24 | SEQ ID NO: 33 |
| (3) | SEQ ID NO: 24 | SEQ ID NO: 34 |
| (4) | SEQ ID NO: 25 | SEQ ID NO: 32 |
| (5) | SEQ ID NO: 25 | SEQ ID NO: 33 |
| (6) | SEQ ID NO: 25 | SEQ ID NO: 34 |
| (7) | SEQ ID NO: 26 | SEQ ID NO: 32 |
| (8) | SEQ ID NO: 26 | SEQ ID NO: 33 |
| (9) | SEQ ID NO: 26 | SEQ ID NO: 34 |
| (10) | SEQ ID NO: 27 | SEQ ID NO: 32 |
| (11) | SEQ ID NO: 27 | SEQ ID NO: 33 |
| (12) | SEQ ID NO: 27 | SEQ ID NO: 34 |
| (13) | SEQ ID NO: 28 | SEQ ID NO: 32 |
| (14) | SEQ ID NO: 28 | SEQ ID NO: 33 |
| (15) | SEQ ID NO: 28 | SEQ ID NO: 34 |
| (16) | SEQ ID NO: 29 | SEQ ID NO: 32 |
| (17) | SEQ ID NO: 29 | SEQ ID NO: 33 |
| (18) | SEQ ID NO: 29 | SEQ ID NO: 34 |
| (19) | SEQ ID NO: 30 | SEQ ID NO: 32 |
| (20) | SEQ ID NO: 30 | SEQ ID NO: 33 |
| (21) | SEQ ID NO: 30 | SEQ ID NO: 34 |
| (22) | SEQ ID NO: 31 | SEQ ID NO: 32 |
| (23) | SEQ ID NO: 31 | SEQ ID NO: 33 |
| (24) | SEQ ID NO: 31 | SEQ ID NO: 34. |

9. The anti-CD24 antibody or antigen-binding fragment thereof according to claim 7, wherein the antibody or antigen-binding fragment thereof comprises a mouse-derived antibody or antigen-binding fragment thereof, a chimeric antibody or antigen-binding fragment thereof, and/or a humanized antibody or antigen-binding fragment thereof.

10. The anti-CD24 antibody or antigen-binding fragment thereof according to claim 5, further comprising an Fc region, and the Fc region is selected from mouse IgG1, IgG2a, IgG2b and/or IgG3, or from rat IgG1, IgG2a, IgG2b and/or IgG2c.

11. The anti-CD24 antibody or antigen-binding fragment thereof according to claim 7, further comprising an Fc region, and the Fc region is selected from human IgG1, IgG2, IgG3 and/or IgG4.

12. (canceled)

13. (canceled)

14. (canceled)

15. A multifunctional fusion protein comprising the anti-CD24 antibody or antigen-binding fragment thereof of claim 7.

16. The multifunctional fusion protein according to claim 15, which further comprises one or more second antibodies or antigen-binding portions thereof that bind specifically to other antigens.

17. The multifunctional fusion protein according to claim 16, wherein the antigen binding the second antibody or antigen-binding portion thereof is selected from a tumor-associated antigen (TAA) or an immune checkpoint.

18. (canceled)

19. (canceled)

20. Use of the anti-CD24 antibody or antigen-binding fragment thereof claim 2 comprising:
a heavy chain variable region and a light chain variable region, wherein the heavy chain variable region comprises the heavy chain complementary determining regions HCDR1, HCDR2, and HCDR3, and the light chain variable region comprises the light chain complementary determining regions LCDR1, LCDR2, and LCDR3, wherein
(g) HCDR1 of the heavy chain variable region is selected from any amino acid sequence of SEQ ID NOs: 2, 17, or a sequence having at least 80%, 85%, 90%, 91%, 92%, 93%, 94%, 95%, 96%, 97%, 98%, 99% or more identity to any amino acid sequence of SEQ ID NOs: 2, 17, or an amino acid sequence having one or more conserved amino acid mutations in comparison to any amino acid sequence of SEQ ID NOs: 2, 17;
(h) HCDR2 of the heavy chain variable region is selected from any amino acid sequence of SEQ ID NOs: 3, 18, or a sequence having at least 80%, 85%, 90%, 91%, 92%, 93%, 94%, 95%, 96%, 97%, 98%, 99% or more identity to any amino acid sequence of SEQ ID NOs: 3, 18, or an amino acid sequence having one or more conserved amino acid mutations in comparison to any amino acid sequence of SEQ ID NOs: 3, 18;
(i) HCDR3 of the heavy chain variable region is selected from any amino acid sequence of SEQ ID NOs: 4, 19, or a sequence having at least 80%, 85%, 90%, 91%, 92%, 93%, 94%, 95%, 96%, 97%, 98%, 99% or more identity to any amino acid sequence of SEQ ID NOs: 4, 19, or an amino acid sequence having one or more conserved amino acid mutations in comparison to any amino acid sequence of SEQ ID NOs: 4, 19;
(j) LCDR1 of the light chain variable region is selected from any amino acid sequence of SEQ ID NOs: 6, 21, or a sequence having at least 80%, 85%, 90%, 91%, 92%, 93%, 94%, 95%, 96%, 97%, 98%, 99% or more identity to any amino acid sequence of SEQ ID NOs: 6, 21, or an amino acid sequence having one or more conserved amino acid mutations in comparison to any amino acid sequence of SEQ ID NOs: 6, 21;
(k) LCDR2 of the light chain variable region is selected from any amino acid sequence of SEQ ID NOs: 7, 22, or a sequence having at least 80%, 85%, 90%, 91%, 92%, 93%, 94%, 95%, 96%, 97%, 98%, 99% or more identity to any amino acid sequence of SEQ ID NOs: 7, 22, or an amino acid sequence having one or more conserved amino acid mutations in comparison to any amino acid sequence of SEQ ID NOs: 7, 22; and/or
(l) LCDR3 of the light chain variable region is selected from any amino acid sequence of SEQ ID NOs: 8, 23, or a sequence having at least 80%, 85%, 90%, 91%, 92%, 93%, 94%, 95%, 96%, 97%, 98%, 99% or more identity to any amino acid sequence of SEQ ID NOs: 8, 23, or an amino acid sequence having one or more conserved amino acid mutations in comparison to any amino acid sequence of SEQ ID NOs: 8, 23, and
and the multifunctional fusion protein claim 15 in the preparation of a drug for the treatment of cancer.

21. The use according to claim 20, wherein the cancer is breast cancer, ovarian cancer, lung cancer, liver cancer, melanoma, malignant glioma, head and neck cancer, colorectal cancer, gastric cancer, bladder cancer, pancreatic cancer, colon cancer, cervical cancer, or a related tumor.

22. (canceled)

23. (canceled)


SEQUENCE LISTING (36 sequences)

SEQ ID NO: 1 - protein, 112 aa, Artificial Sequence
  VH
QIQLVQSGPELKKPGETVKISCKASGYIFTDYAIHWVKQAPGKGLKWMGFINTATGEPTYADDFKGRFAFSLETSASTAYLQVNSLKNEDTATYFCTSGVHWGQGTTLTVSS

SEQ ID NO: 2 - protein, 5 aa, Artificial Sequence
  HCDR1
DYAIH

SEQ ID NO: 3 - protein, 17 aa, Artificial Sequence
  HCDR2
FINTATGEPTYADDFKG

SEQ ID NO: 4 - protein, 3 aa, Artificial Sequence
  HCDR3
GVH

SEQ ID NO: 5 - protein, 106 aa, Artificial Sequence
  VL
QIVLTQSPAIMSASLGEEITLTCSASSSVSSMHWYQQKSGTSPKLLIYSTSNLASGVPSRFSGSGSGTFYSLTISSVESEDAADYYCHHWTSYMYTFGGGTKLEIK

SEQ ID NO: 6 - protein, 10 aa, Artificial Sequence
  LCDR1
SASSSVSSMH

SEQ ID NO: 7 - protein, 7 aa, Artificial Sequence
  LCDR2
STSNLAS

SEQ ID NO: 8 - protein, 9 aa, Artificial Sequence
  LCDR3
HHWTSYMYT

SEQ ID NO: 9 - protein, 112 aa, Artificial Sequence
  VH
QVQLVQSGSELKKPGASVKVSCKASGYIFTDYAIHWVRQAPGQGLEWMGFINTATGEPTYADDFKGRFVFSLDTSVSTAYLQISSLKAEDTAVYYCARGVHWGQGTLVTVSS

SEQ ID NO: 10 - protein, 112 aa, Artificial Sequence
  VH
QVQLVQSGSELKKPGASVKVSCKASGYIFTDYAIHWVRQAPGQGLEWMGFINTATGEPTYADDFKGRFVFSLDTSVSTAYLQISSLKAEDTAVYFCTSGVHWGQGTLVTVSS

SEQ ID NO: 11 - protein, 112 aa, Artificial Sequence
  VH
QVQLVQSGSELKKPGASVKVSCKASGYIFTDYAIHWVKQAPGQGLEWMGFINTATGEPTYADDFKGRFVFSLDTSVSTAYLQISSLKAEDTAVYFCTSGVHWGQGTLVTVSS

SEQ ID NO: 12 - protein, 112 aa, Artificial Sequence
  VH
QVQLVQSGSELKKPGASVKVSCKASGYIFTDYAIHWVKQAPGQGLKWMGFINTATGEPTYADDFKGRFVFSLDTSVSTAYLQISSLKAEDTAVYFCTSGVHWGQGTLVTVSS

SEQ ID NO: 13 - protein, 106 aa, Artificial Sequence
  VL
DIQMTQSPSTLSASVGDRVTITCSASSSVSSMHWYQQKPGKAPKLLIYSTSNLASGVPSRFSGSGSGTEFTLTISSLQPDDFATYYCHHWTSYMYTFGGGTKVEIK

SEQ ID NO: 14 - protein, 106 aa, Artificial Sequence
  VL
DIQMTQSPSTLSASVGDRVTITCSASSSVSSMHWYQQKPGKAPKLLIYSTSNLASGVPSRFSGSGSGTEYTLTISSLQPDDFATYYCHHWTSYMYTFGGGTKVEIK

SEQ ID NO: 15 - protein, 106 aa, Artificial Sequence
  VL
DIQLTQSPSTLSASVGDRVTITCSASSSVSSMHWYQQKPGKSPKLLIYSTSNLASGVPSRFSGSGSGTEYTLTISSLQPDDFATYYCHHWTSYMYTFGGGTKVEIK

SEQ ID NO: 16 - protein, 117 aa, Artificial Sequence
  VH
EVQLQESGPDLVKPSQSLSLTCTVTGYSITSGYTWHWIRQFPGNKLEWMGHIQYTGNTKYNPSLESRISITRETSKNQFFLQLNSVTTEDTGTYYCARGSTNSNDYWGQGTSVTVSS

SEQ ID NO: 17 - protein, 6 aa, Artificial Sequence
  HCDR1
SGYTWH

SEQ ID NO: 18 - protein, 16 aa, Artificial Sequence
  HCDR2
HIQYTGNTKYNPSLES

SEQ ID NO: 19 - protein, 8 aa, Artificial Sequence
  HCDR3
GSTNSNDY

SEQ ID NO: 20 - protein, 113 aa, Artificial Sequence
  VL
DIVMTQSPSSLAVSVGEKVTVSCKSSQSLLYSNDQKNYLAWYQQKPGQSPKLLIYWASTRESGVPDRFTGSGSGTDFTLTITSVKAEDLAVYYCQQYFIYPLTFGAGTKLELK

SEQ ID NO: 21 - protein, 17 aa, Artificial Sequence
  LCDR1
KSSQSLLYSNDQKNYLA

SEQ ID NO: 22 - protein, 7 aa, Artificial Sequence
  LCDR2
WASTRES

SEQ ID NO: 23 - protein, 9 aa, Artificial Sequence
  LCDR3
QQYFIYPLT

SEQ ID NO: 24 - protein, 117 aa, Artificial Sequence
  VH
QVQLQESGPGLVKPSQTLSLTCTVSGYSITSGYTWHWIRQHPGKGLEWIGHIQYTGNTKYNPSLESRVTISVDTSKNQFSLKLSSVTAADTAVYYCARGSTNSNDYWGQGTLVTVSS

SEQ ID NO: 25 - protein, 117 aa, Artificial Sequence
  VH
QVQLQESGPGLVKPSQTLSLTCTVSGYSITSGYTWHWIRQHPGKGLEWIGHIQYTGNTKYNPSLESRVTISVDTSKNQFSLKLSSVTAADTATYYCARGSTNSNDYWGQGTLVTVSS

SEQ ID NO: 26 - protein, 117 aa, Artificial Sequence
  VH
QVQLQESGPGLVKPSQTLSLTCTVSGYSITSGYTWHWIRQHPGNGLEWIGHIQYTGNTKYNPSLESRVTISVDTSKNQFSLKLSSVTAADTATYYCARGSTNSNDYWGQGTLVTVSS

SEQ ID NO: 27 - protein, 117 aa, Artificial Sequence
  VH
QVQLQESGPGLVKPSQTLSLTCTVSGYSITSGYTWHWIRQHPGNKLEWIGHIQYTGNTKYNPSLESRVTISVDTSKNQFSLKLSSVTAADTATYYCARGSTNSNDYWGQGTLVTVSS

SEQ ID NO: 28 - protein, 117 aa, Artificial Sequence
  VH
QVQLQESGPGLVKPSQTLSLTCTVSGYSITSGYTWHWIRQFPGNKLEWIGHIQYTGNTKYNPSLESRVTISVDTSKNQFSLKLSSVTAADTATYYCARGSTNSNDYWGQGTLVTVSS

SEQ ID NO: 29 - protein, 117 aa, Artificial Sequence
  VH
EVQLQESGPGLVKPSQTLSLTCTVSGYSITSGYTWHWIRQFPGNKLEWIGHIQYTGNTKYNPSLESRVTISVDTSKNQFSLKLSSVTAADTATYYCARGSTNSNDYWGQGTLVTVSS

SEQ ID NO: 30 - protein, 117 aa, Artificial Sequence
  VH
EVQLQESGPGLVKPSQTLSLTCTVSGYSITSGYTWHWIRQFPGNKLEWIGHIQYTGNTKYNPSLESRITISRDTSKNQFSLKLSSVTAADTATYYCARGSTNSNDYWGQGTLVTVSS

SEQ ID NO: 31 - protein, 117 aa, Artificial Sequence
  VH
EVQLQESGPGLVKPSQTLSLTCTVSGYSITSGYTWHWIRQFPGNKLEWMGHIQYTGNTKYNPSLESRITISRDTSKNQFSLKLSSVTAADTATYYCARGSTNSNDYWGQGTLVTVSS

SEQ ID NO: 32 - protein, 113 aa, Artificial Sequence
  VL
DIVMTQSPDSLAVSLGERATINCKSSQSLLYSNDQKNYLAWYQQKPGQPPKLLIYWASTRESGVPDRFSGSGSGTDFTLTISSLQAEDVAVYYCQQYFIYPLTFGGGTKLELK

SEQ ID NO: 33 - protein, 113 aa, Artificial Sequence
  VL
DIVMTQSPDSLAVSLGERATINCKSSQSLLYSNDQKNYLAWYQQKPGQSPKLLIYWASTRESGVPDRFSGSGSGTDFTLTISSLQAEDVAVYYCQQYFIYPLTFGGGTKLELK

SEQ ID NO: 34 - protein, 113 aa, Artificial Sequence
  VL
DIVMTQSPSSLAVSLGERATINCKSSQSLLYSNDQKNYLAWYQQKPGQSPKLLIYWASTRESGVPDRFSGSGSGTDFTLTISSLQAEDVAVYYCQQYFIYPLTFGGGTKLELK

SEQ ID NO: 35 - protein, 330 aa, Artificial Sequence [sequence omitted]

SEQ ID NO: 36 - protein, 107 aa, Artificial Sequence [sequence omitted]